(19) United States
(12) Patent Application Publication
(10) Pub. No.: US 2011/0245212 A1
(43) Pub. Date: Oct. 6, 2011

(54) Methods of alleviating the symptoms of premenstrual syndrome/late luteal phase dysphoric disorder

(76) Inventor: John M. Masi, (US)

(21) Appl. No.: 12/755,230

(22) Filed: Apr. 6, 2010

(51) Int. Cl. A61K 31/585; A61P 5/24

(52) U.S. Cl. 514/171

(57) ABSTRACT

The present invention is a method of a non-continuous administration of a pharmaceutical to a female for a condition associated with the female's menstrual cycle, compromising in the administration of either 7α-Acetylthio-3-oxo-17α-pregn-4-ene-21,17-carbolactone, (+)-(S)-2-(6-methoxynaphthalen-2-yl) propanoic acid or RS)-2-(4-(2-methylpropyl) phenyl) propanoic acid. The active ingredients to be manufactured into a tablet or chewable formulation rich in cocoa flavonols such as dark chocolate no more than 65%. A method of inhibiting one or more symptom of premenstrual syndrome/late luteal phase dysphoric disorder compromising of administering to a female in need of treatment an effective amount of a compound. The first dose to be given at the onset of the premenstrual symptoms. This can be given two to four times a day up to seven days as directed. This is intended to be a short course of therapy to alleviate the symptoms of premenstrual syndrome/late luteal phase dysphoric disorder.

### REFERENCES CITED (REFERENCED BY)

U.S. Patent Documents

| | | |
|---|---|---|
| 5,733,937 | February 1997 | MacLean et al. |
| 20050176693 | February 2005 | Boissonneault, Roger M. et al. |
| 5,441,986 | August 1995 | Thompson |
| 5,389,670 | February 1995 | Fontana, Steven |
| 5,047,431 | September 1991 | Schickaneder et al. |
| 5,254,594 | October 1993 | Kazuaki et al. |
| 5,384,332 | January 1995 | Fontana, Steven |
| 4,888,343 | December 1989 | Jones, Howard et al. |
| 6,077,530 | July 1997 | Weinstein, Robert and Allan |

### BACKGROUND OF THE INVENTION

Each month, for the first few days prior to the onset of menstruation, many millions otherwise healthy women develop symptoms of disturbed mood and appetite that can be strikingly similar to those reported by patients with Seasonal Affective Disorder (SAD), carbohydrate craving obesity, or the non anorexic variants of bulimia. This syndrome was first termed “premenstrual tension” by R. T. Frank in 1931 and is a very common phenomenon. According to Guy Abraham of UCLA, of every ten patients to walk into a gynecologist's office, three or four will suffer from premenstrual tension and, in some the symptoms will be of such severity as to include attempts at suicide. Current Progress in Obstetrics and Gynecology, 3:5-39 (1980). Some suggest that up to 75-80% of women exhibit some symptoms of PMS during their childbearing years. PMS is usually hits the hardest around the time frame of 30-49 years of age.

Initial descriptions of the Premenstrual Syndrome (PMS) focused on its association with nervous tension, headache and weight gain. The weight gain observed was initially attributed to excessive retention of salt and water, which does indeed occur in some PMS patients. However, it soon became evident that it was also a consequence of the widespread tendency of some individuals suffering from PMS to crave and over consume some carbohydrates, particularly foods with a sweet taste such as chocolates. It is believed that carbohydrates and chocolate in particular may cause the brain to release serotonin. PMS is also now referred to as late luteal phase dysphoric disorder. D. N. S. III, Revised American Psychiatric Association (1987)

There have been numerous suggestions made about the etiology of PMS. For example, some hypothesized that it was caused by a uterine toxin. Others have suggested its cause was over consumption of sweets, which was presumably followed by excessive insulin secretion, hypoglycemia, and inadequate brain glucose, and resulted in the often observed depression and anxiety. It has also been postulated that the behavioral symptoms result from tissue edema often observed and that the psychological changes result from feelings of loss or the social complexities generated by the discomfort of menstruation.

However, none of these theories has been substantiated: PMS can persist after hysterectomy, and, hence uterine toxins cannot be its cause; the hyperinsulinism of PMS is not associated with low blood glucose levels, and it is probably the consequence of a behavioral aberration (i.e., the tendency of premenstrual women to choose high carbohydrate diets, which potentiate insulin secretion) rather than the cause; the mood and appetite changes of PMS are poorly correlated with the tissue swelling; and subhuman primates who are presumably exempt from the psychodynamic or social complexities of human life also exhibit characteristic behavioral changes premenstrually.

There have been many treatments suggested for overcoming or reducing the symptoms of PMS. These include carbohydrate free diets, vitamin supplements, and ovarian hormones, detoxifying agents, selective serotonin reuptake inhibitors, thiazide type diuretics and oral contraceptives.

Late Luteal Phase Dysphoric Disorder (LLPDD) is the current term associated with Premenstrual Syndrome (PMS). Many females report a variety of physical and emotional changes associated with specific phases of the menstrual cycle. For most of these females, these changes are not severe, cause little distress, and have no effect on social or occupational functioning. In contrast the essential feature of LLPDD is a pattern of clinically significant emotional and behavioral symptoms that occur during the last week of the luteal phase and remit within a few days after the onset of the follicular phase. In most females, these symptoms occur in the week before and remit within a few days after the onset of menses.

Among the most commonly experienced symptoms are marked affective lability (e.g., sudden episodes of tearfulness, sadness, or irritability), persistent feelings of irritability, anger, or tension, feelings of depression, and self deprecating thoughts. Also common are decreased interest in usual activities, fatigability and loss of energy, a subjective sense of difficulty in concentration, changes in appetite, craving for specific foods (especially carbohydrates and chocolate), and sleep disturbances. Other physical symptoms such as breast tenderness or swelling, headaches, joint or muscle pain, a sensation of bloating, and weight gain, also may be present.

Generally, non-steroidal anti-inflammatory drugs are administered to LLPDD patients, but these only are effective for some of the physical symptoms. The physical manifestation of PMS, if severe, may be treated symptomatically. Water retention may be relieved by diet or antidiuretic medication, but severity of water retention does not always correlate with psychological symptoms. Recent studies have suggested that spironolactone may also be effective in relieving depression and crying spells. Other drugs, including progesterone, lithium carbonate, thiazide, diuretics, antidepressants and bromocriptine have been tried with limited success.

### SUMMARY OF INVENTION

The present invention relates is a method of non-continuous administration of a pharmaceutical to a human female for inhibiting the symptoms of PMS/LLPDD. These symptoms are usually associated with a females menstrual cycle. The invention is intended to be a short course of therapy lasting no longer than seven days. This short course of therapy can be repeated monthly.

The model of this invention adheres to a method of non-continuous administration of a pharmaceutical to a women for a condition associated with PMS or the late luteal phase of a female's menstrual cycle. The daily oral dosage intended to be given two to four times day, starting on the first day of the onset of symptoms, prior to menses and continuing until the symptoms are alleviated, but not to exceed seven days.

There are a few products containing a non-steroidal anti-inflammatory such as aspirin, or ibuprofen that is marketed for use in treating late luteal phase dysphoric disorder and or premenstrual syndrome. These products have the disadvantage in that they do not contain a highly potent diuretic, another factor which is important in relieving PMS/LLPDD. These products may also contain caffeine, cinnamedrine or pamabrom. Cinnamedrine is conventionally recognized as an anti-spasmodic. Caffeine is recognized in these products as a diuretic, Pamabrom is an antihistamine. Using caffeine in these products can increase irritability and sleeplessness in women who are already suffering from a painful episode of PMS/LLPDD.

The present invention involves a composition which contains essentially as the only pharmaceutically active ingredients (a) a non-steroidal anti-inflammatory selected from the group consisting of either ibuprofen or naproxen, and (b) a diuretic from the group of aldosterone antagonist, these ingredients in a base of cocoa flavonols (dark chocolate) being present at a level in these compositions to afford relief from the symptoms late luteal phase dysphoric disorder, or premenstrual syndrome when the said compositions are administered to women exhibiting symptoms associated with those conditions. These products have the virtue of containing effective anti-inflammatory agents and a potent diuretic which not only relieves water retention but offers the additional benefit of relieving depression, breast tenderness, and crying spells. The composition of these pharmaceutically active ingredients in a tablet or chewable tablet that uses dark chocolate in small doses in its base satisfies their cravings for carbohydrates and sweet foods, and potentially helps foster the release of serotonin.

As indicated above the non-steroidal anti-inflammatory agents that are useful for the present purposes are ibuprofen or naproxen. These drugs are never used in combination since they are similarly like each other. The quantity of said non-steroidal anti-inflammatory that will be contained in the present composition may be expressed in the form of the daily average for these agents. The daily average for each non-steroidal anti-inflammatory will vary with each drug. Generally for ibuprofen this will be from 100 mg-2000 mg with the preferred range from 200 mg to 1600 mg. In the case of naproxen the general average will be about 100 mg to 1000 mg. the preferred range being 220 mg-880 mg.

In similar fashion, the quantity of the particular diuretics of use in the present invention and contained in the present products may also be expressed on the basis of the daily average dose for spironolactone. In this case the daily average dose of spironolactone will be from 25 mg-100 mg. In both cases whether it is used with ibuprofen or naproxen the range of spironolactone will still be 25 mg-100 mg.

The active ingredients of this invention will generally be administered in a convenient chewable tablet dosage form that is in a base containing cocoa flavonols no more than 65% of dark chocolate. Dark chocolate has many medicinal properties due to its level of flavonols. In small doses the dark chocolate will satiate the cravings for sweets and carbohydrates.

The product to be packaged in a blister pack clearly marked days one through seven. Each day will be marked am dose and pm dose with respect to naproxen and spironolactone and the amount of suggested doses to be taken. The ibuprofen and spironolactone will be marked breakfast, lunch, late afternoon, and evening, also with the suggested doses to be taken.

The various dosage units are each provided as chewable tablets. The formulation may be swallowed, or it may be a sucked or chewed as intended. The dosage units may further include conventional excipients well known in the pharmaceutical industry such as binding agents, gallants, fillers, lubricants, disintergrants, surfactants, and colorants, and for suckable or chewable tablets.

Thus, in a preferred embodiment the invention provides dosage unit containers distinguishing between the various dosages, including instructions on how the medications should be taken. Examples of the two different formulations of ibuprofen and or naproxen with spironolactone in a dark chocolate base in addition to the other binding agents are as follows.

### EXAMPLE 1

The two chewable AM unit dosage (ingredients) of Naproxen 220 mg+Spironolactone 50 mg will be in a dark chocolate base. The two chewable evening unit dosage of Naproxen 220 mg will be in a dark chocolate base.

### EXAMPLE 2

The two chewable AM unit dosage (ingredients) of Naproxen 220 mg+Spironolactone 50 mg will be in a dark chocolate base. The two chewable evening unit dosages of Naproxen 220 mg+Spironolactone 25 mg will be in a dark chocolate base.

### EXAMPLE 3

The two chewable AM unit dosage (ingredients) of Naproxen 220 mg+Spironolactone 50 mg will be in a dark chocolate base. The two chewable evening unit dosages of Naproxen 220 mg+Spironolactone 50 mg will be in a dark chocolate base.

### EXAMPLE 4

Two chewable AM unit dosage (ingredients) of Ibuprofen 200 mg+Spironolactone 25 mg in a dark chocolate base. The lunch dose would consist of two chewable tablets of Ibuprofen 200 mg in a dark chocolate base. He late afternoon dosage would be the same as the AM dosage, and the evening dosage would be the same as the lunch regimen.

### EXAMPLE 5

Two chewable AM unit dosage (ingredients) of Ibuprofen 200 mg+Spironolactone 50 mg in a dark chocolate base. The lunch dose would consist of two chewable tablets of Ibuprofen 200 mg in a dark chocolate base. He late afternoon dosage would be the same as the AM dosage, and the evening dosage would be the same as the lunch regimen.

What is claimed is:

1. A pharmaceutical composition for the relief of alleviating the symptoms of premenstrual syndrome/late luteal phase dysphoric disorder. This compound will consist of either (a) ibuprofen, or (b) naproxen with (c) spironolactone as the pharmaceutically active ingredients in a cocoa base rich in flavonols. These pharmaceutically active ingredients (a) and (c) or (b) and (c) being present in the composition in amounts effective to afford relief from the symptoms of PMS/late luteal phase dysphoric disorder and or premenstrual syndrome.

2. A pharmaceutical composition according to claim 1 including a pharmaceutical carrier.

3. A pharmaceutical composition according to claim 1 having as an average daily dose for ibuprofen from 200 mg to 2400 mg of ibuprofen and from about 25 mg to 200 mg of spironolactone.

4. A pharmaceutical composition according to claim 1 having as an average daily dose from about 100 mg to about 1000 mg of naproxen, with about 25 mg to 200 mg of spironolactone.

5. A pharmaceutical composition according to claim 3 in which the average daily dose for ibuprofen is from 200 mg-1600 mg, and an average daily dose for spironolactone is 25 mg-200 mg.

6. A pharmaceutical composition according to claim 4 in which the average daily dose for naproxen is from 220 mg-880 mg, and the average daily dose for spironolactone is 25 mg-200 mg.

7. A pharmaceutical composition according to claim 1 in unit dosage form containing 100 mg-200 mg of ibuprofen, and about 12.5 mg-25 mg of spironolactone.

8. A pharmaceutical composition according to claim 1 in unit dosage form containing 110 mg-220 mg of naproxen, and about 12.5 mg-25 mg of spironolactone.

9. A pharmaceutical composition according to claim 1, having an average daily dose of about 100 mg to 2000 mg of ibuprofen given with 12.5 mg to 200 mg of spironolactone.

10) A pharmaceutical composition according to claim 1 having an average daily dose from about 62.5 mg to 1000 mg of naproxen given with 12.5 mg to 200 mg of spironolactone.

11) A pharmaceutical composition according to claim 1 being selected from a group consisting of an aldosterone antagonist, and a non steroidal anti-inflammatory drug.

12) A pharmaceutical composition according to claim 1, where the active ingredients will be incorporated into a chewable tablet formulation consisting of a base rich in cocoa flavonols also known as dark chocolate.

13) According to claim 1, the said pharmaceutical dosage period is a short course therapy lasting no longer than 7 days.

14) According to claim 1 the intended compound will be used for premenstrual syndrome which is premenstrual dysphoric disorder or late luteal phase dysphoric disorder.

15) According to claim 12, the dark chocolate will contain at least 50% cocoa, but not greater than 80%.

16) The pharmaceutical dosage according to claims 7 and 8, will be prepackaged in pre-packed single dosage units given two to four times a day.

17) According to claim 13, the total number of days to administer the pre-packed single dosage units will be 7 days.

18) According to claim 13, the short course of therapy lasting no longer than 7 days, can be repeated monthly at the onset of symptoms prior to menses.

19) A process for relieving symptoms of premenstrual syndrome in a patient exhibiting such symptoms which compromises administering to the patient a composition of claims 1, 2, 3, 4, 5, 6, 7, 8, 9, 10, 11, 12, 13, 14, 15, 16, 17, and 18.
